(19) United States
(12) Patent Application Publication
(10) Pub. No.: US 2009/0287220 A1
(43) Pub. Date: Nov. 19, 2009

(54) PERCUTANEOUS SCREW EXTRACTOR

(75) Inventor: P. Douglas Kiester, Irvine, CA (US)

Correspondence Address: P. DOUGLAS KIESTER;CBRK ENTERPRISES, 19 Rippling Stream, Irvine, CA, 92603, US

(73) Assignee: CBRK ENTERPRISES, Irvine, CA (US)

(21) Appl. No.: 12/271,739

(22) Filed: Nov. 14, 2008

Related U.S. Application Data
(60) Provisional application No. 60/988,268, filed on Nov. 15, 2007.

(51) Int. Cl. A61B 17/58

(52) U.S. Cl. 606/104

(57) ABSTRACT

Apparatus and methods for percutaneous screw extraction from orthopedic plates are described. The screw extractor eliminates the need for relatively large incisions to expose an orthopedic plate for the replacement of failed screws in the plate. The present invention further provides a device to function with slightly modified screws that allows the screws to be removed quickly, easily, and safely, with no larger incisions than were used to install the plate.

### CROSS-REFERENCE TO RELATED APPLICATIONS

This application claims the benefit of U.S. provisional application No. 60/988,268, filed Nov. 15, 2007.

### BACKGROUND OF THE INVENTION

The present invention generally relates to orthopedic plate and screw combinations and, more particularly, to apparatus and methods for replacement of failed screws in orthopedic plates.

Recently there have been great strides in plate fixation of human bone fractures. Among the more important of these developments is the locking plate.

A conventional orthopedic plate does not firmly connect to the fixation screws. Rather, the screws loosely pass through the plate in almost any direction. The screw head puts pressure on the plate which then presses on the bone to hold the construct in plate. With this construct, the screws can fail one at a time and pull straight out.

With a locking plate, the head of the screw screws into the plate. Thus the screw is firmly fixed to the plate. The screw angle to the plate cannot be changed once the screw is in place. Now, the screw cannot fail by pulling straight out in most fractures and all the screws must often fail at the same time if there is a failure.

Methods now exist to make a small incision and then run a locking plate up along the bone, without exposing the bone. The locking screws are placed percutaneously through small skin incisions.

A problem occurs if the plate needs to be removed. Current methods require the entire length of the plate to be opened with an incision.

Another problem with screw removal is scar tissue. This improved somewhat when hex-head (Allen wrench type) screws are used in place of standard screws. Most locking screws now use a torx head.

As can be seen, there is a need for improved orthopedic plate and screw combinations, especially those combinations that allow for percutaneous removal of the screws from the plates.

### SUMMARY OF THE INVENTION

In one aspect of the present invention, a screw extractor comprises a tip having at least two beads of threads about at least a portion thereof; a locking portion adjacent an end of the tip for locking into a screw.

In another aspect of the present invention, a screw and extractor combination comprises a screw having a torx head; a tip on the extractor, the tip having a tip cross sectional area smaller than a screw head cross sectional area; and a locking portion on the extractor for locking to the torx head.

In a further aspect of the present invention, a method for extracting a screw comprises creating an incision in a skin layer; inserting a tip of a screw extractor through the incision; engaging the tip in a head of the screw; locking the tip to grooves in the screw with teeth adjacent an end of the tip.

These and other features, aspects and advantages of the present invention will become better understood with reference to the following drawings, description and claims.

### BRIEF DESCRIPTION OF THE DRAWINGS

FIG. 1 is a perspective view of a screw extractor according to the present invention, positioned above a locking plate;

FIG. 2 is a close-up side perspective view of a screw extractor according to the present invention;

FIG. 3 is a close-up end perspective view of the screw extractor of FIG. 2;

FIG. 4 is a close-up end view of the screw extractor of FIG. 2;

FIG. 5 is a plan view of a screw according to the present invention;

FIG. 6 is a side perspective view of the screw of FIG. 5;

FIGS. 7a through 7e describe a method of using the screw extractor of the present invention;

FIG. 8 is a side view of a quick release handle usable on the screw extractor of the present invention; and

FIGS. 9A through 9E show the use of a trochar and working tube with the screw extractor of the present invention.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

Broadly, the present invention generally provides apparatus and methods for percutaneous screw extraction from orthopedic plates. In contrast to previous apparatus and methods, the present invention eliminates the need for relatively large incisions to expose an orthopedic plate for the replacement of failed screws in the plate. Also in contrast to prior apparatus and methods, the present invention provides a device to function with slightly modified screws that allows the screws to be removed quickly, easily, and safely, with no larger incisions than were used to install the plate.

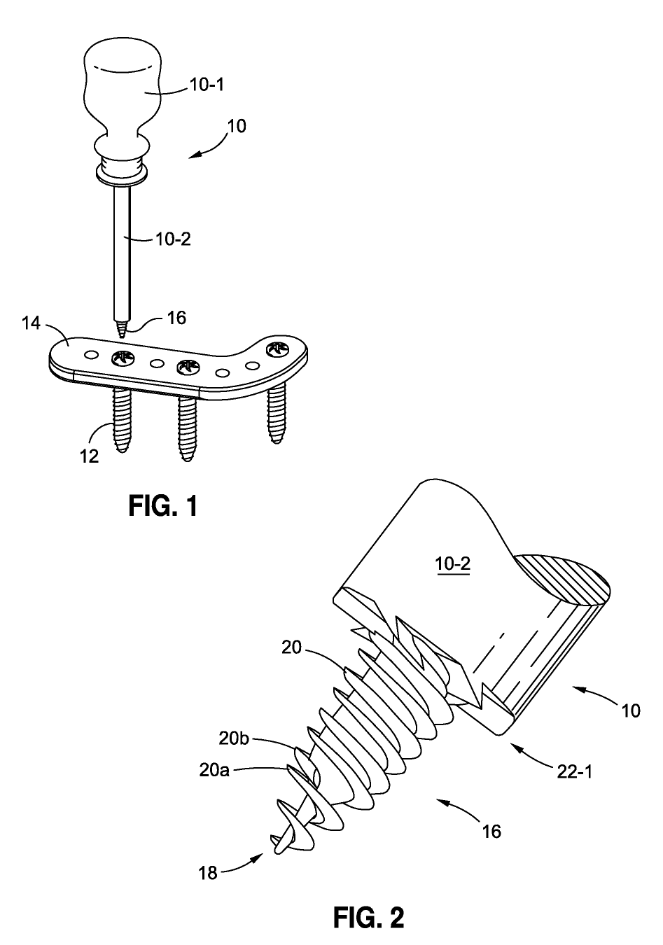

Referring to FIG. 1, there is shown a perspective view of a screw extractor 10 according to the present invention, positioned above a screw 12 positioned in a locking plate 14. The screw extractor 10 may include a handle 10-1, a shaft 10-2 and a tip 16. While the present invention, as described herein, shows the use of locking plates 14, the present invention may be used with any type of orthopedic plate.

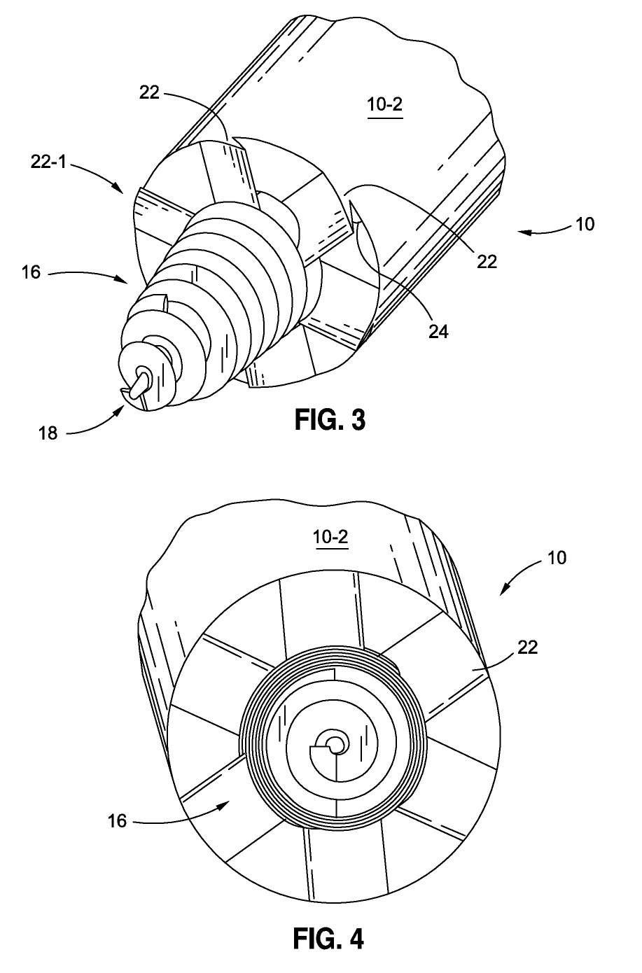

Referring to FIGS. 2 through 4, there are shown views of the tip 16 of the screw extractor 10 of the present invention. The present invention provides, in one example, the screw extractor 10 which may have a small tip 16 and taper toward its distal end 18. This may allow the surgeon to feel (under x-ray control) when the tip 16 of the screw extractor 10 has found the middle of the head of the screw 12.

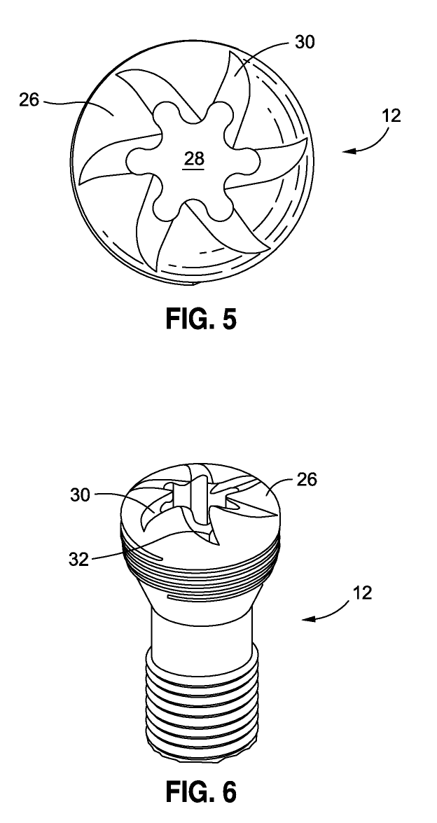

The screw extractor 10 may have a reverse-threaded corkscrew design, wherein threads 20 undulate about the tip 16 in a reverse-threaded direction. When the screw extractor 10 is inserted into the screw 12, this design may pull the screw extractor 10 into the torx head of the screw 12, as described in more detail below. The tip 16 of the screw extractor 10 may be designed so it does not fill the torx head of the screw 12 completely, thereby either cutting through the scar tissue (which may be present when attempting screw removal) or compressing it out of the way. For removal of the screw 12 using a conventional torx screwdriver (not shown), the torx opening 28 (see FIG. 5) may become plugged with scar tissue, preventing the torx screwdriver to engage the torx opening 28. Even if the torx screwdriver is able to engage the torx opening 28, after loosening the screw 12 from the screw plate 14 (see FIG. 1), there is no attachment between the torx screwdriver and the screw 12 to permit pulling the screw 12 out of the patient. The threaded tip 16 of the present invention may overcome these problems.

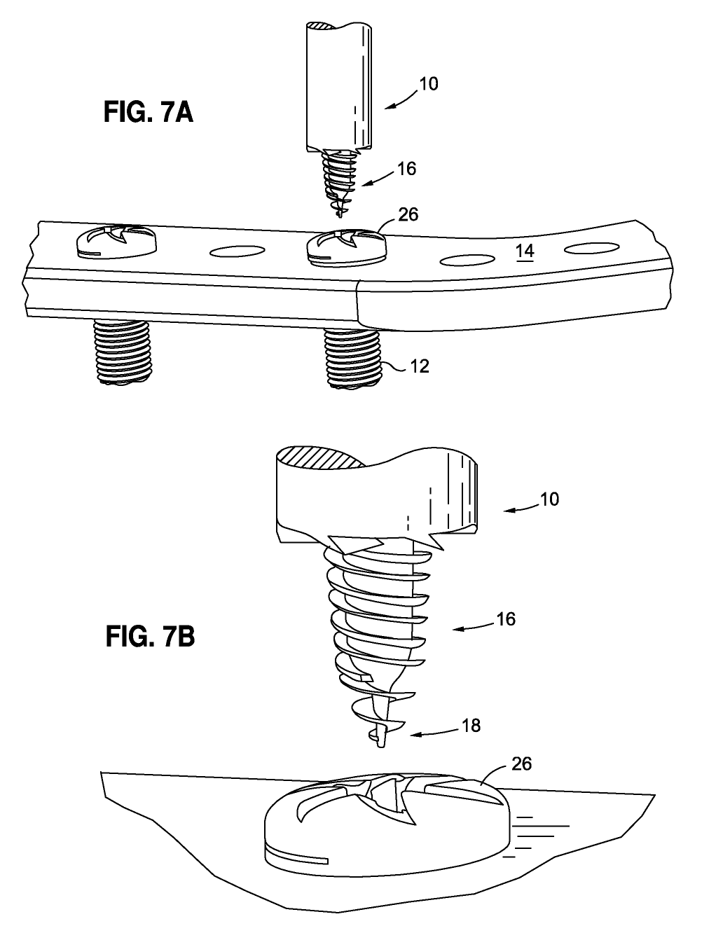

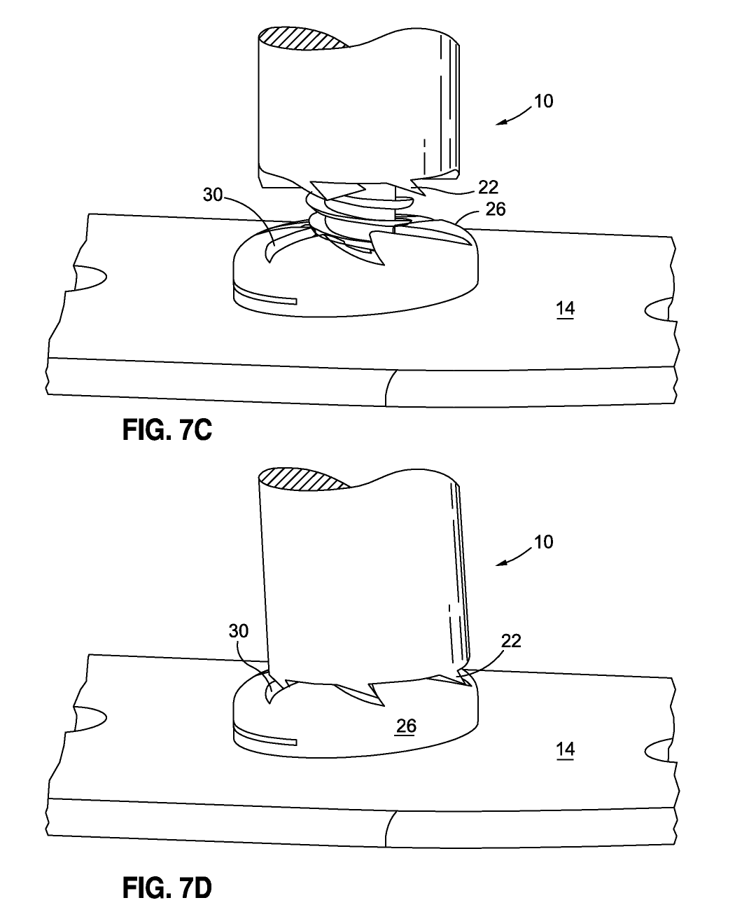

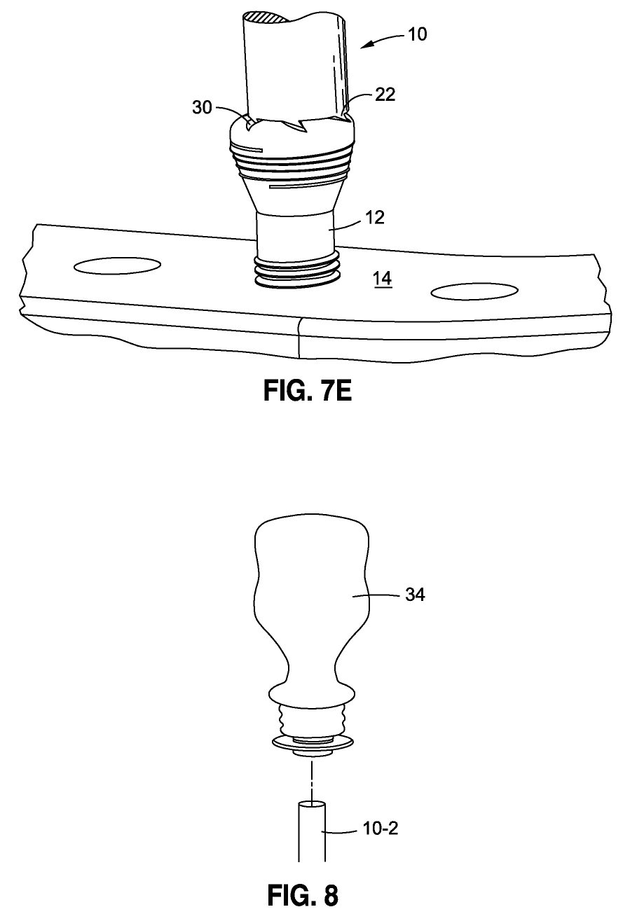

The tip 16 of the screw extractor 10 may have a portion of the threads 20 that are double beaded. In other words, instead of a single thread 20 undulating about the tip 16, in at least a portion of the tip 16, two threads 20a, 20b may be present. This may cause the screw extractor 10 to advance faster (with fewer turns), allowing the teeth 22 of the screw extractor (described below) to have a large enough angle 24 to bite into the top of the screw 12 during extraction thereof. This aspect of the present invention is described in more detail below with respect to FIGS. 7a through 7e.

At the end of the shaft 10-2 of the screw extractor 10, a locking portion 22-1, including teeth 22 may be present. The teeth 22 may depend from the shaft 10-2 at an angle 24 toward the tip 16. The teeth 22 may be designed to fit into the screw 12, as described below with respect to FIGS. 5 and 6.

Referring now to FIGS. 5 and 6, there are shown views of the screw 12 usable with the screw extractor 10 of the present invention. The head 26 of the screw 12 may have a standard torx opening 28 wherein a torx screwdriver (not shown) may be used to install the screw. The head 26 may have grooves 30 cut therein to align with the teeth 22 of the screw extractor 10. The grooves 30 may be cut into the head 26 at an angle 32 at an angle similar (for example, within 2 to 10 degrees) to the angle 24 of the teeth 22 depending from the screw extractor 10.

While FIGS. 5 and 6, as well as the steps shown below with respect to FIGS. 7a through 7e, describe the screw head 26 having grooves 30 cut therein, screws 12 without grooves 30 may also be useful with the screw extractor 10 of the present invention. In this case, the teeth 22 of the screw extractor 10 may press against the head 26 of the screw 12. In one embodiment, the teeth 22 of the screw extractor may cut into the screw 12, forming grooves in the screw 12 during extraction thereof.

Referring to FIGS. 7a through 7e, there is shown a method for using the screw extractor 10 of the present invention. FIG. 7a shows the tip 16 of the screw extractor 10 almost inserted into the head 26 of the screw 12 installed in the plate 14. FIG. 7b is a close-up view of this alignment taking place.

In FIG. 7c, the threads 20 may cut into the torx (or hex, for example) screw head 26 as the screw extractor 10 is turned counter-clockwise on the screw 12. This may allow the teeth 22 of the screw extractor 10 to approach the grooves 30 of the screw head 26.

In FIG. 7d, the screw extractor 10 is shown fully engaged with the screw 12. The teeth 22 may fit into the grooves 30 as shown in FIG. 7d. Pressure from the scar tissue (not shown) and the top of the screw 12 locked into the screw extractor 10 can now give enough pressure on the screw 12 to unlock it from the plate 14.

In FIG. 7e, as the screw extractor 10 is further turned (after full engagement with the screw 12), the screw 12 may be removed from the plate 14 and/or bone (not shown). In should be appreciated that the screw extractor 10 may remain engaged with the screw 12 while the screw 12 is pulled from the plate 14, through the soft tissues and out from the subject.

Referring to FIG. 8, there is shown a side view of a handle 34 usable on the screw extractor 10 of the present invention. The handle 34 may be a quick release handle, as is known in the art, allowing for quick removal of the handle and permitting attachment of a tool (not shown), such as a drill, for completion of the screw removal.

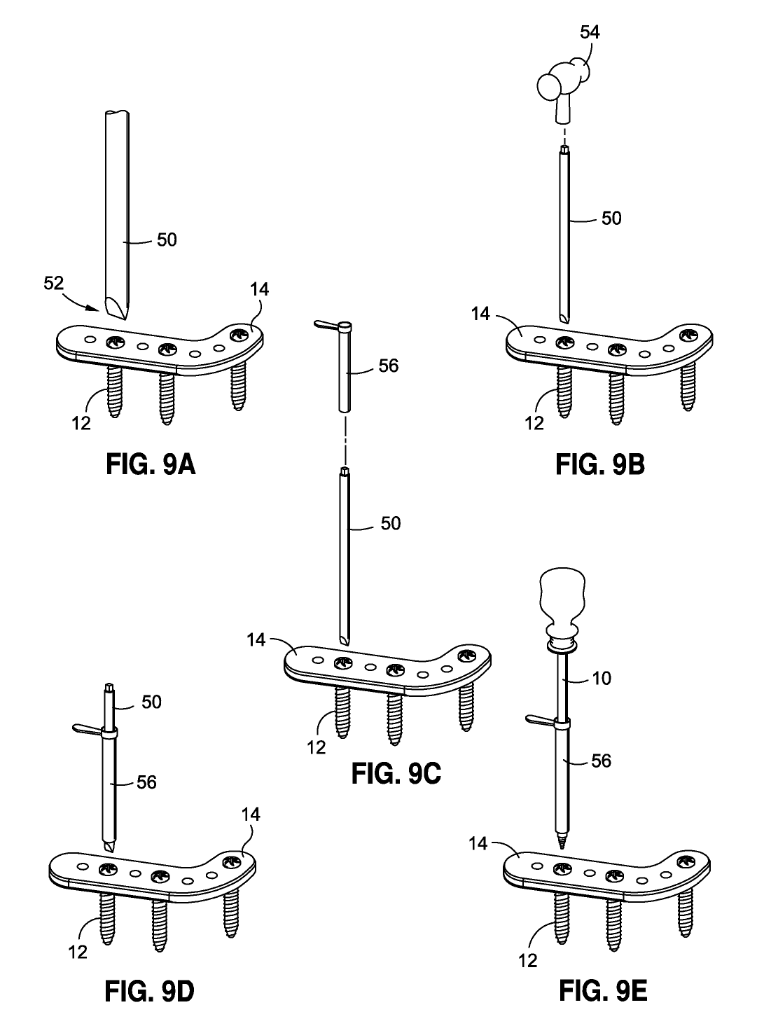

Referring to FIGS. 9A through 9E, a trochar 50 may be used to gain access to the screw 12. The trochar 50 may have a tip 52 shaped like a chisel for guiding the trochar 50 through the soft tissue (not shown) to gain access to the screw 12. Furthermore, the chisel-like tip 52 may also be useful for scraping off soft tissues form the area of the plate 14 near the screw 12. A handle 54 of the trochar 50 may be removable to allow a working tube 56 to be slid down the trochar 50 and guided to the plate 14, as shown in FIGS. 9C and 9D. The trochar 50 may then be removed and other implements, such as the screw extractor 10 may be used by insertion through the working tube 56. For example, FIG. 9E shows the screw extractor 10 being guided to the screw 12 through the working tube 56.

It should be understood, of course, that the foregoing relates to exemplary embodiments of the invention and that modifications may be made without departing from the spirit and scope of the invention as set forth in the following claims.

I claim:

1. A screw extractor, comprising:
a tip having at least two beads of threads about at least a portion thereof;
a locking portion adjacent an end of the tip for locking onto a screw.

2. The screw extractor of claim 1, wherein the locking portion includes teeth depending from a shaft of the screw extractor toward the tip of the screw extractor.

3. The screw extractor of claim 2, wherein the teeth are spaced to fit into grooves formed in a head of the screw.

4. The screw extractor of claim 3, wherein the number of teeth equal the number of grooves.

5. The screw extractor of claim 2, wherein the screw lacks grooves and the teeth press against a head of the screw.

6. The screw extractor of claim 2, wherein screw lacks grooves and the teeth cut into a head of the screw, thereby engaging the screw and forming grooves in the screw during extraction thereof.

7. The screw extractor of claim 1, wherein the tip cross sectional area is smaller than a screw head opening cross sectional area.

8. The screw extractor of claim 1, further comprising a quick release handle on an end of the screw extractor opposite the tip.

9. A screw and extractor combination, comprising:
a screw; and
a tip on the extractor, the tip threading into the screw, the tip having at least two beads of threads about at least a portion thereof.

10. The combination of claim 9, wherein the screw has a hex-shaped head.

11. The combination of claim 9, wherein the screw has a torx head and the tip has a tip cross sectional area smaller than a torx opening cross sectional area of the torx head.

12. The combination of claim 9, further comprising a locking portion on the extractor for locking to the screw.

13. The combination of claim 12, wherein the locking portion includes teeth depending from the extractor toward the tip thereof.

14. The combination of claim 13, wherein the screw has a head with grooves cut therein.

15. The combination of claim 14, wherein the grooves are designed to engage with the teeth and the extractor is used with the screw to extract the screw.

16. A method for extracting a screw, comprising:
creating an incision in a skin layer;
inserting a tip of a screw extractor through the incision;
engaging the tip in a head of the screw; and
threading the tip into the screw.

17. The method of claim 16, further comprising locking the tip into the screw with teeth adjacent an end of the tip.

18. The method of claim 17, wherein the tip has at least two beads of threads about at least a portion thereof.

19. The method of claim 18, wherein the tip has a tip cross sectional area smaller than a torx opening cross sectional area.

20. The method of claim 16, further comprising removing a handle of the screw extractor and attaching a drill thereto.

21. The method of claim 16, further comprising removing the screw from a subject while maintaining engagement between the screw extractor and the screw.
